(19) United States
(12) Patent Application Publication
(10) Pub. No.: US 2010/0004494 A1
(43) Pub. Date: Jan. 7, 2010

(54) CONVERSION OF METHANE INTO C3˜C13 HYDROCARBONS

(75) Inventors: Wensheng Li, Hunan (CN); Li Huang, Hunan (CN); Yanqun Ren, Hunan (CN); Xiaoping Zhou, Hunan (CN)

Correspondence Address: BAKER & MCKENZIE LLP, Pennzoil Place, South Tower, 711 Louisiana, Suite 3400, HOUSTON, TX, 77002-2716, US

(73) Assignee: MICROVAST TECHNOLOGIES, LTD., Huzhou, Zhejiang (CN)

(21) Appl. No.: 12/293,663

(22) PCT Filed: Mar. 12, 2007
(86) PCT No.: PCT/CN07/00780

§ 371 Date: Aug. 7, 2009

(30) Foreign Application Priority Data
Mar. 20, 2006 (CN) 200610031377.9

(51) Int. Cl. C07C 1/26

(52) U.S. Cl. 585/310

(57) ABSTRACT

A process for preparing C₃˜C₁₃ hydrocarbons from methane, oxygen and HBr/H₂O is provided including the steps of reacting methane with oxygen and HBr/H₂O over a first catalyst in a first reactor to form CH₃Br and CH₂Br₂; converting CH₃Br and CH₂Br₂ into C₃˜C₁₃ hydrocarbons and HBr over a second catalyst in a second reactor; and recovering the HBr produced in the second reactor.

### FEDERALLY SPONSORED RESEARCH

Not applicable.

### REFERENCE TO MICROFICHE APPENDIX

Not applicable.

### FIELD OF THE INVENTION

The present invention relates to a novel process for preparing C₃˜C₁₃ hydrocarbons from methane. This invention is an extension of application CN200410022850.8 and relates to the following research results in more depth and detail.

### BACKGROUND OF THE INVENTION

Natural gas is the most abundant hydrocarbon resource on earth besides coal, and is mainly composed of methane with a small amount of other compounds such as ethane, propane, steam, and carbon dioxide. Compared with coal, natural gas is a cleaner hydrocarbon resource because it can be directly used as fuel or chemical feedstock to produce other chemical products. Since most natural gas resources are often discovered in remote areas and natural gas is difficult to compress and transport, the cost to use natural gas is quite high. On the other hand, the high stability of C—H bonds of methane makes the chemical conversion difficult. In currently available technologies, natural gas is mostly used to make hydrogen or synthesis gas (H₂+CO) (also referred to as “syngas”). With the hydrogen being used to produce ammonia, and the syngas converted to methanol. Although the Fischer-Tropsch method can convert natural gas into fuel oil through a syngas process, the cost is higher than that of original petroleum refining method. Therefore, natural gas is not widely used as a substitute for petroleum to produce fuel oil or other chemical monomers. A new process for converting methane into easily transported liquid petroleum or other synthesis intermediates is thus desired. Since the syngas route is not a cost-effective process, it has been suggested to produce higher value chemicals from light alkanes by selective oxidation processes. Except for a few successful examples such as preparing maleic anhydride by oxidation of n-butane, most cases of selective oxidation method of light alkanes, such as CH₄, C₂H₆ and C₃H₈, did not achieve successful application in chemical industry because of low conversion rate, low selectivity, and difficulty to separate the products.

Another method involves converting methane into methanol [Roy A., Periana et al., Science, 280, 560(1998)] and acetic acid [Roy A. Periana, et al., Science, 301, 814(2003)]. In such process, SO₂ was produced that could not be recovered, and concentrated sulphuric acid, which was used as reactant and solvent, was diluted after the reaction and could not be used continuously. This method has not been industrialized.

In the earlier paper [G. A. Olah et al. Hydrocarbon Chemistry(Wiley, New York,1995)], Olah reported the process to form CH₃Br and HBr by reacting methane and Br₂, then to hydrolyze CH₃Br to provide methanol and dimethyl ether. This report did not suggest or disclose how to recycle HBr. The object of such process was not to synthesize hydrocarbons, and the reported single-pass conversion rate of methane was lower than 20%. The inventors of the present invention had also designed a process to convert alkane to methanol and dimethyl ether (Xiao Ping Zhou et al., Chem Commun. 2294(2003); Catalysis Today 98, 317(2004).; U.S. Pat. No. 6,486,368; U.S. Pat. No. 6,472,572; U.S. Pat. No. 6,465,696; U.S. Pat. No. 6,462,243). Such process, however, related to the use of Br₂ and the extra step of regenerating Br₂. As known, the utilization and storage of vast amount of Br₂ is very dangerous.

### SUMMARY OF THE INVENTION

In some embodiments of the invention, a process for preparing C₃˜C₁₃ hydrocarbons from methane, oxygen and HBr/H₂O is provided including the steps of reacting methane with oxygen and HBr/H₂O over a first catalyst in a first reactor to form CH₃Br and CH₂Br₂; converting CH₃Br and CH₂Br₂ into C₃˜C₁₃ hydrocarbons and HBr over a second catalyst in a second reactor; and recovering the HBr produced in the second reactor.

### DETAILED DESCRIPTION OF EMBODIMENTS OF THE INVENTION

In the process of the present invention, methane is converted into alkyl bromides and then the alkyl bromides are further converted into corresponding products. Meanwhile, HBr is collected and directed into the first reactor for reuse. This process has wide application in preparing chemicals. Embodiments of the present inventive process are energy-saving. For example, when gasoline is prepared by the inventive process, the two exothermic reactions included in the inventive process can be carried out under atmospheric pressure. In embodiments of the inventive process, the raw materials for preparing alkyl bromides are O₂, natural gas and HBr/H₂O, in which HBr/H₂O solution are used as bromine source instead of Br₂, and the use of HBr/H2O offers a much safer solution to overall process because the reactions are strong exothermic, and H2O from HBr/H2O can carry significant heat away. Thus, the temperature of the catalytic bed can be easily controlled. In embodiments of the present invention, HBr is regenerated in the process of converting alkyl bromides into hydrocarbons. Embodiments of the present do not require a separate step to regenerate Br₂.

One aim of some embodiments of the present invention is to efficiently convert methane of natural gas into liquid hydrocarbons or easily-liquefied hydrocarbons.

Embodiments of the inventive process include two reactions shown below.
A: methane reacts with HBr/H₂O and O₂ to form alkyl bromides:

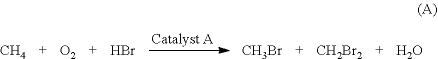

B: alkyl bromides are converted into higher hydrocarbons and HBr by the catalyst B.

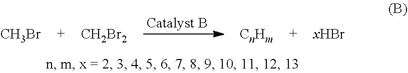

HBr can be reused in the reaction A to complete one cycle.

### EXAMPLES

### Examples 1-23

### Oxidative Bromination of Alkanes

The catalysts were prepared as follows: Silica (10 g, S_{BET}=1.70 m²/g), RuCl₃ solution (0.00080 g Ru/mL) and corresponding metal nitrates solution (0.10M) were mixed in a mole ratio of components of catalysts given in Table 1, stirred at ambient temperature for 0.5 h, dried at 110° C. for 4 h, and then calcined at 450° C. for 12 h.

The catalytic reaction was carried out in the quartz-tube reactor (i.d. 0.80 cm, length 60 cm) at the temperatures shown in Table 1, packed with 1.0000 g catalyst with both ends filled with quartz sand, with reactant flows: 5.0 mL/min of methane, 5.0 mL/min of oxygen, 4.0 mL (liquid)/h of 40 wt % HBr/H₂O solution. The products were analyzed by a gas chromatography. Results are set forth in Table 1.

TABLE 1
Components of Catalysts, Temperature and Results of the Reactions

| | | | Conversion | | | | |
|---|---|---|---|---|---|---|---|
| | Temperature | | Rate | Selectivity (mol %) | | | |
| Sample | (° C.) | Catalysts | (mol %) | CH₃Br | CH₂Br₂ | CO | CO₂ |
| 1 | 580 | 0.1%Ru/SiO₂ | 38.4 | 52.9 | 0 | 47.1 | 0 |
| 2 | 580 | 0.1%Rh/SiO₂ | 35.9 | 37.9 | 0 | 62.1 | 0 |
| 3 | 580 | 5%Mg0.1%Ru/SiO₂ | 32.1 | 53.1 | 4.5 | 42.4 | 0 |
| 4 | 580 | 5%Ca0.1%Ru/SiO₂ | 20.9 | 33.1 | 3.3 | 63.6 | 0 |
| 5 | 580 | 5%Ba0.1%Ru/SiO₂ | 25.9 | 76.8 | 6.6 | 16.6 | 0 |
| 6 | 580 | 5%Y0.1%Ru/SiO₂ | 69.9 | 15.4 | 1.8 | 77.7 | 5.1 |
| 7 | 580 | 5%La0.1%Ru/SiO₂ | 72.2 | 30.7 | 5.6 | 61.0 | 2.7 |
| 8 | 580 | 5%Sm0.1%Ru/SiO₂ | 81.4 | 7.6 | 2.1 | 86.9 | 3.4 |
| 9 | 600 | 5%Sm0.1%Ru/SiO₂ | 86.6 | 6.8 | 1.2 | 88.0 | 4.0 |
| 10 | 580 | 2.5%Ba2.5%La0.1%Ru/SiO₂ | 42.9 | 55.9 | 6.1 | 38.0 | 0 |
| 11 | 580 | 2.5%Ba2.5%La/SiO₂ | 15.7 | 52.2 | 14.6 | 33.2 | 0 |
| 12 | 600 | 2.5%Ba2.5%La0.1%Ru/SiO₂ | 58.8 | 53.4 | 4.9 | 41.7 | 0 |
| 13 | 580 | 2.5%Ba2.5%Sm0.1%Ru/SiO₂ | 34.5 | 61.8 | 9.1 | 29.1 | 0 |
| 14 | 600 | 2.5%Ba2.5%Sm0.1%Ru/SiO₂ | 41.5 | 57.2 | 5.0 | 37.8 | 0 |
| 15 | 580 | 2.5%Ba2.5%Bi0.1%Ru/SiO₂ | 18.2 | 60.2 | 16.2 | 23.6 | 0 |
| 16 | 600 | 2.5%Ba2.5%Bi0.1%Ru/SiO₂ | 37.1 | 49.9 | 5.8 | 44.3 | 0 |
| 17 | 600 | 2.5%Ba2.5%La0.5%Bi0.1%Ru/SiO₂ | 50.0 | 54.4 | 7.0 | 38.6 | 0 |
| 18 | 600 | 2.5%Ba2.5%La0.5%Fe0.1%Ru/SiO₂ | 59.3 | 51.7 | 3.1 | 40.4 | 4.8 |
| 19 | 600 | 2.5%Ba2.5%La0.5%Co0.1%Ru/SiO₂ | 52.1 | 52.2 | 3.4 | 38.2 | 6.2 |
| 20 | 600 | 2.5%Ba2.5%La0.5%Ni0.1%Ru/SiO₂ | 62.9 | 54.5 | 5.3 | 34.6 | 5.6 |
| 21 | 600 | 2.5%Ba2.5%La0.5%Cu0.1%Ru/SiO₂ | 41.3 | 51.4 | 2.8 | 39.4 | 6.4 |
| 22 | 600 | 2.5%Ba2.5%La0.5%V0.1%Ru/SiO₂ | 57.6 | 50.5 | 3.0 | 38.0 | 8.5 |
| 23 | 600 | 2.5%Ba2.5%La0.5%Mo0.1%Ru/SiO₂ | 53.6 | 52.1 | 2.4 | 36.0 | 9.5 |

Notes:
methane: 5.0 mL/min, oxygen: 5.0 mL/min, 40 wt % HBr/H₂O: 4.0 mL (liquid)/h, catalyst: 1.0000 g

### Example 24

The catalysts were prepared as follows: Silica (10 g, S_{BET}=0.50 m²/g), RuCl₃ solution (0.00080 g Ru/mL), La(NO₃)₃ solution (0.01M), Ba(NO₃)₂ solution (0.10M), Ni(NO₃)₂ solution (0.10M) were mixed in a mole ratio of 2.5% La, 2.5% Ba, 0.5% Ni, 0.1% Ru and 94.4% SiO₂. The mixture was stirred at ambient temperature for 0.5 h, dried at 110° C. for 4 h, and then calcined at 450° C. for 12 h to give the catalyst with composition as La2.5% Ba2.5% Ni0.5% Ru0.1%/SiO₂.

The catalytic reaction was carried out in the quartz-tube reactor (i.d. 1.50 cm, length 60 cm) at 660° C., packed with 5.000 g catalyst with both ends filled with quartz sand, with reactant flows: 15.0 mL/min of methane, 5.0 mL/min of oxygen, 6.0 mL(liquid)/h of 40 wt % HBr/H₂O solution. The products were analyzed by a gas chromatography. Methane conversion rate was 32.0%, and the selectivity of CH₃Br, CH₂Br₂, CO and CO₂ were 80.8%, 0.67%, 15.7% and 2.9%, respectively.

### Examples 25-38

### Conversion From Alkane Bromide to Hydrocarbons

### Preparation of Catalyst ZnO/HZSM-5 and MgO/HZSM-5

The catalysts C1-C14 of example 25-38 in Table 2 were prepared as follows: HZSM-5 (Si/Al=360, 283 m²/g), water and Zn(NO₃)₂.6H₂O (or Mg(NO₃)₂.6H₂O) were mixed in a ratio given in Table 2 and stirred and impregnated at ambient temperature for 12 h, dried at 120° C. for 4 h, and then calcined at 450° C. for 8 h. The catalyst was tabletted at 100 atm pressure, and then crushed and sieved to 40-60 mesh to the catalysts shown in Table 2.

TABLE 2

| | | | HZSM-5 | H₂O | Mg(NO₃)₂•6H₂O | Zn(NO₃)₂•6H₂O |
|---|---|---|---|---|---|---|
| Sample | Catalyst | Component | (g) | (mL) | (g) | (g) |
| 25 | C1 | 5.0wt%ZnO/HZSM-5 | 10.0000 | 30.0 | 0 | 1.8276 |
| 26 | C2 | 6.0wt%ZnO/HZSM-5 | 10.0000 | 30.0 | 0 | 2.1931 |
| 27 | C3 | 8.0wt%ZnO/HZSM-5 | 10.0000 | 30.0 | 0 | 2.9242 |
| 28 | C4 | 10.0wt%ZnO/HZSM-5 | 10.0000 | 30.0 | 0 | 3.6522 |
| 29 | C5 | 12.0wt%ZnO/HZSM-5 | 10.0000 | 30.0 | 0 | 4.3862 |
| 30 | C6 | 14.0wt%ZnO/HZSM-5 | 10.0000 | 30.0 | 0 | 5.1173 |
| 31 | C7 | 15.0wt%ZnO/HZSM-5 | 10.0000 | 30.0 | 0 | 5.4828 |
| 32 | C8 | 5.0wt%MgO/HZSM-5 | 10.0000 | 30.0 | 3.2051 | 0 |
| 33 | C9 | 6.0wt%MgO/HZSM-5 | 10.0000 | 30.0 | 3.2051 | 0 |
| 34 | C10 | 8.0wt%MgO/HZSM-5 | 10.0000 | 30.0 | 5.1281 | 0 |
| 35 | C11 | 10.0wt%MgO/HZSM-5 | 10.0000 | 30.0 | 6.4102 | 0 |
| 36 | C12 | 12.0wt%MgO/HZSM-5 | 10.0000 | 30.0 | 7.6922 | 0 |
| 37 | C14 | 14.0wt%MgO/HZSM-5 | 10.0000 | 30.0 | 8.9743 | 0 |
| 38 | C14 | 15.0wt%MgO/HZSM-5 | 10.0000 | 30.0 | 9.6153 | 0 |

The catalysts of example 25-38 were used to convert CH₃Br into hydrocarbons. The reaction was carried out in the glass-tube reactor (i.d. 1.50 cm) with 8.0 g catalyst at 240° C., with a flow of 6.8 mL/min of CH₃Br. The products were analyzed by a gas chromatography. The conversion rate of CH₃Br and the selectivity of hydrocarbons are set forth in Table 3. Cₙ in Table 3 means the total amount of alkanes containing n carbons.

TABLE 3
Conversion Rate of CH₃Br and Product Selectivity

| | Alkanes and Alkenes | | | | | | | | | Aromatics | | | | | | |
|---|---|---|---|---|---|---|---|---|---|---|---|---|---|---|---|---|
| | X | C₂ | C₃ | C₄ | C₅ | C₆ | C₇ | C₈ | C₉ | C₇ | C₈ | C₉ | C₁₀ | C₁₁ | C₁₂ | C₁₃ |
| Catalyst | (%) | (%) | (%) | (%) | (%) | (%) | (%) | (%) | (%) | (%) | (%) | (%) | (%) | (%) | (%) | (%) |
| C1 | 91.0 | 2.8 | 15.3 | 44.2 | 20.9 | 9.7 | 3.4 | 0.0 | 0.2 | 0.1 | 0.5 | 1.6 | 0.7 | 0.2 | 0.3 | 0.1 |
| C2 | 97.4 | 1.6 | 12.2 | 44.0 | 21.6 | 10.4 | 3.8 | 0.7 | 0.3 | 0.1 | 1.0 | 2.6 | 1.0 | 0.3 | 0.3 | 0.1 |
| C3 | 98.3 | 1.6 | 13.7 | 42.2 | 18.9 | 9.3 | 4.8 | 1.2 | 0.3 | 0.1 | 1.3 | 4.0 | 1.5 | 0.4 | 0.6 | 0.1 |
| C4 | 98.7 | 1.6 | 9.1 | 33.0 | 22.2 | 19.0 | 4.3 | 1.2 | 0.4 | 0.2 | 1.4 | 4.3 | 1.8 | 0.5 | 0.8 | 0.2 |
| C5 | 95.4 | 1.9 | 12.0 | 42.4 | 21.4 | 12.7 | 3.1 | 0.3 | 0.1 | 0.0 | 0.3 | 1.1 | 4.4 | 0.1 | 0.2 | 0.0 |
| C6 | 94.4 | 1.9 | 15.5 | 47.6 | 19.4 | 7.6 | 2.7 | 0.6 | 0.2 | 0.1 | 0.7 | 2.2 | 0.9 | 0.2 | 0.3 | 0.1 |
| C7 | 92.0 | 1.8 | 14.9 | 44.7 | 20.9 | 10.9 | 4.4 | 0.3 | 0.1 | 0.0 | 0.3 | 1.0 | 0.4 | 0.1 | 0.2 | 0.0 |
| C8 | 99.6 | 1.9 | 10.9 | 45.9 | 20.5 | 11.1 | 3.6 | 0.7 | 0.5 | 0.3 | 1.1 | 0.5 | 0.8 | 1.2 | 0.4 | 0.6 |
| C9 | 99.6 | 2.6 | 9.4 | 44.3 | 22.4 | 12.5 | 5.5 | 0.7 | 0.4 | 0.0 | 0.7 | 0.3 | 0.3 | 0.5 | 0.2 | 0.2 |
| C10 | 99.6 | 3.3 | 5.7 | 49.2 | 27.9 | 4.7 | 6.3 | 0.6 | 0.4 | 0.0 | 0.6 | 0.2 | 0.6 | 0.3 | 0.1 | 0.1 |
| C11 | 99.6 | 2.9 | 7.5 | 44.6 | 22.8 | 10.5 | 4.3 | 0.9 | 0.5 | 0.3 | 1.9 | 0.8 | 0.9 | 1.3 | 0.5 | 0.3 |
| C12 | 99.3 | 2.5 | 8.5 | 39.6 | 24.7 | 12.0 | 5.9 | 1.1 | 0.5 | 0.0 | 1.5 | 0.6 | 1.7 | 0.8 | 0.5 | 0.1 |
| C13 | 99.6 | 3.3 | 5.7 | 49.1 | 26.7 | 4.1 | 6.3 | 0.9 | 0.5 | 0.0 | 0.9 | 0.4 | 0.7 | 0.7 | 0.2 | 0.5 |
| C14 | 99.5 | 2.0 | 6.9 | 46.5 | 25.5 | 10.0 | 4.2 | 0.9 | 0.5 | 0.2 | 1.0 | 0.4 | 0.6 | 0.7 | 0.5 | 0.1 |

Note:
X means the conversion rate of CH₃Br.

### Examples 39-53

The catalysts C15-C29 of example 39-53 in Table 4 were prepared as follows: (Si/Al=360, 283 m²/g), water and corresponding salts were mixed in a ratio given in Table 4 and stirred and impregnated at ambient temperature for 12 h, dried at 120° C. for 4 h, and then calcined at 450° C. for 8 h. The catalyst was tabletted at 100 atm pressure, and then crushed and sieved to 40-60 mesh to the catalysts shown in Table 4.

TABLE 4

| | | | | | Second | HZSM-5 |
|---|---|---|---|---|---|---|
| Sample | Catalyst | Catalyst | First composition | | composition | (g) |
| 39 | C15 | Co/HZSM-5 | CoCl₂•6H₂O | 1.5877 g | H₂O 30 ml | 10.000 |
| 40 | C16 | Cr/HZSM-5 | Cr(NO₃)3•9H₂O | 1.3160 g | H₂O 30 ml | 10.000 |
| 41 | C17 | Cu/HZSM-5 | CuCl₂•2H₂O | 1.0722 g | H₂O 30 ml | 10.000 |
| 42 | C18 | Ca/HZSM-5 | Ca(NO₃)₂•4H₂O | 2.1085 g | H₂O 30 ml | 10.000 |
| 43 | C19 | Fe/HZSM-5 | Fe(NO₃)₃•9H₂O | 2.5250 g | H₂O 30 ml | 10.000 |
| 44 | C20 | Ag/HZSM-5 | AgNO₃ | 0.7322 g | H₂O 30 ml | 10.000 |
| 45 | C21 | Pb/HZSM-5 | Pb(NO₃)₂ | 0.7426 g | H₂O 30 ml | 10.000 |
| 46 | C22 | Bi/HZSM-5 | Bi(NO₃)₃•5H₂O | 1.0413 g | H₂O 30 ml | 10.000 |
| 47 | C23 | Ce/HZSM-5 | Ce(NO₃)₂•6H₂O | 1.3229 g | H₂O 30 ml | 10.000 |
| 48 | C24 | Sr/HZSM-5 | Sr(NO₃)₂ | 1.0212 g | H₂O 30 ml | 10.000 |
| 49 | C25 | La/HZSM-5 | La(NO₃)₃•6H₂O | 1.3291 g | H₂O 30 ml | 10.000 |
| 50 | C26 | Y/HZSM-5 | Y(NO₃)₃•6H₂O | 1.6963 g | H₂O 30 ml | 10.000 |
| 51 | C27 | Mn/HZSM-5 | MnCl₂ | 1.3800 g | H₂O 30 ml | 10.000 |
| 52 | C28 | Nb/HZSM-5 | NbCl₅ | 1.0514 g | C₂H₅OH 40 ml | 10.000 |
| 53 | C29 | Ti/HZSM-5 | TiCl₄ | 1.000 ml | C₂H₅OH 40 ml | 10.000 |

The catalysts of example 39-53 were used to convert CH₃Br into hydrocarbons. The reaction was carried out in the glass-tube reactor (i.d. 1.50 cm) with 8.0 g catalyst at 200-240° C., with a flow of 6.8 mL/min of CH₃Br. The products were analyzed by a gas chromatography. The conversion rate of CH₃Br and the selectivity of hydrocarbons are given in Table 5. Cₙ in Table 5 means the total amount of alkanes containing n carbons.

TABLE 5
Conversion Rate of CH₃Br and Product Selectivity

| | | T | X | C2 | C3 | C4 | C5 | C6 | C7 |
|---|---|---|---|---|---|---|---|---|---|
| Catalyst | Catalyst | (° C.) | (%) | (%) | (%) | (%) | (%) | (%) | (%) |
| C15 | Co/HZSM-5 | 240 | 84.9 | 4.7 | 10.8 | 32.6 | 18.1 | 17.2 | 16.6 |
| C16 | Cr/HZSM-5 | 200 | 44.0 | 0 | 13.6 | 73.8 | 12.6 | 0 | 0 |
| C16 | Cr/HZSM-5 | 220 | 79.8 | 6.8 | 15.6 | 45.2 | 14.6 | 8.5 | 9.4 |
| C16 | Cr/HZSM-5 | 240 | 81.1 | 9.3 | 16.9 | 36.1 | 22.9 | 8.6 | 6.2 |
| C17 | Cu/HZSM-5 | 200 | 62.7 | 0 | 11.6 | 52.7 | 22.2 | 13.4 | 0 |
| C17 | Cu/HZSM-5 | 220 | 67.5 | 4.4 | 25.2 | 45.8 | 16.6 | 4.5 | 3.5 |
| C17 | Cu/HZSM-5 | 240 | 71.1 | 1.8 | 7.0 | 22.1 | 60.3 | 4.2 | 4.6 |
| C18 | Ca/HZSM-5 | 220 | 94.8 | 0 | 13.8 | 44.4 | 15.3 | 17.1 | 9.4 |
| C18 | Ca/HZSM-5 | 240 | 95.0 | 0 | 21.3 | 49.5 | 17.6 | 6.8 | 4.9 |
| C19 | Fe/HZSM-5 | 200 | 39.7 | 8.2 | 8.6 | 41.1 | 18.4 | 16.7 | 7.0 |
| C19 | Fe/HZSM-5 | 220 | 75.6 | 12.0 | 20.2 | 45.0 | 10.1 | 12.7 | 0 |
| C19 | Fe/HZSM-5 | 240 | 69.6 | 25.9 | 20.8 | 32.2 | 11.3 | 4.8 | 5.0 |
| C20 | Ag/HZSM-5 | 200 | 24.6 | 0 | 10.9 | 29.2 | 27.1 | 15.3 | 17.4 |
| C20 | Ag/HZSM-5 | 220 | 50.9 | 25.9 | 20.8 | 32.2 | 11.3 | 4.8 | 5.0 |
| C20 | Ag/HZSM-5 | 240 | 70.0 | 0 | 14.7 | 56.8 | 22.4 | 2.5 | 3.7 |
| C21 | Pb/HZSM-5 | 220 | 70.1 | 25.9 | 20.7 | 32.2 | 11.2 | 4.9 | 5.1 |
| C21 | Pb/HZSM-5 | 240 | 82.6 | 7.7 | 14.9 | 32.3 | 19.5 | 12.6 | 13.5 |
| C22 | Bi/HZSM-5 | 200 | 33.8 | 6.1 | 7.1 | 30.3 | 23.2 | 30.6 | 2.6 |
| C23 | Ce/HZSM-5 | 200 | 70.6 | 2.9 | 4.2 | 22.9 | 25.8 | 14.5 | 29.6 |
| C23 | Ce/HZSM-5 | 220 | 76.3 | 0 | 10.9 | 29.2 | 27.1 | 15.3 | 17.4 |
| C23 | Ce/HZSM-5 | 240 | 77.0 | 25.9 | 20.8 | 32.2 | 11.3 | 4.8 | 5.0 |
| C24 | Sr/HZSM-5 | 200 | 62.5 | 11.2 | 4.4 | 36.7 | 39.2 | 1.3 | 7.0 |
| C24 | Sr/HZSM-5 | 220 | 85.9 | 6.8 | 15.6 | 45.2 | 14.6 | 8.5 | 9.4 |
| C24 | Sr/HZSM-5 | 240 | 98.1 | 9.3 | 16.9 | 36.1 | 22.9 | 8.6 | 6.2 |
| C25 | La/HZSM-5 | 200 | 63.7 | 2.9 | 4.2 | 22.9 | 25.8 | 14.5 | 29.6 |
| C25 | La/HZSM-5 | 220 | 70.8 | 0 | 10.9 | 29.2 | 27.1 | 15.3 | 17.4 |
| C25 | La/HZSM-5 | 240 | 75.8 | 25.9 | 20.8 | 32.2 | 11.3 | 4.8 | 5.0 |
| C26 | Y/HZSM-5 | 200 | 13.3 | 0 | 6.7 | 36.6 | 29.1 | 18.3 | 9.2 |
| C26 | Y/HZSM-5 | 220 | 64.2 | 3.8 | 23.5 | 39.8 | 19.7 | 9.8 | 3.3 |
| C26 | Y/HZSM-5 | 240 | 69.2 | 5.4 | 11.9 | 42.5 | 24.4 | 10.6 | 5.1 |
| C27 | Mn/HZSM-5 | 200 | 67.0 | 7.1 | 14.0 | 39.4 | 24.5 | 10.3 | 4.6 |
| C27 | Mn/HZSM-5 | 240 | 83.7 | 3.4 | 6.5 | 37.9 | 26.4 | 13.0 | 12.7 |
| C28 | Nb/HZSM-5 | 200 | 68.5 | 3.2 | 17.1 | 40.5 | 22.1 | 10.4 | 6.5 |
| C28 | Nb/HZSM-5 | 240 | 68.5 | 3.6 | 5.9 | 30.9 | 23.0 | 15.2 | 21.4 |
| C29 | Ti/HZSM-5 | 220 | 46.8 | 4.2 | 13.1 | 41.7 | 23.9 | 10.5 | 6.7 |
| C29 | Ti/HZSM-5 | 240 | 79.2 | 4.9 | 22.1 | 41.6 | 19.4 | 5.6 | 6.5 |

### Example 54

### Reaction-in-Series: Oxidative Bromination of Methane and Hydrocarbons; Conversion from CH₃Br

For preparing the catalyst, Silica (10 g, S_{BET}=0.50 m²/g), RuCl₃ solution (0.00080 g Ru/mL), La(NO₃)₃ solution (0.10 M), Ba(NO₃)₂ solution (0.10 M), Ni(NO₃)₂ solution (0.10 M) were mixed in a mole ratio of 2.5% La, 2.5% Ba, 0.5% Ni, 0.1% Ru and 94.4% SiO₂. The result solution was stirred at ambient temperature for 0.5 h, dried at 110° C. for 4 h, and then calcined at 450° C. for 12 h to give the catalyst with component as La2.5% Ba2.5% Ni0.5% Ru0.1%/SiO₂.

The catalytic reaction was carried out in the quartz-tube reactor (i.d. 1.50 cm, length 60 cm) at 660° C., packed with 5.000 g catalyst with both ends filled with quartz sand, with reactant flows: 15.0 mL/min of methane, 5.0 mL/min of oxygen, 6.0 mL(liquid)/h of 40 wt % HBr/H₂O solution. The products were analyzed by a gas chromatography. Methane conversion rate was 32.0%, and the selectivities of CH₃Br, CH₂Br₂, CO and CO₂ were 80.8%, 0.67%, 15.7% and 2.9%, respectively. The composite undergone first step reaction was directly introduced into glass-tube reactor (i.d. 1.5 cm) at 240° C., which was packed with 8.0 g 14.0 wt % MgO/HZSM-5 catalyst. The final products were analyzed by a gas chromatography. The conversions rate of CH₃Br and CH₂Br₂ were 100% through the second reactor and the products were hydrocarbons of C₂˜C₁₃. The similar result was achieved using 8.0 g 14.0 wt % ZnO/HZSM-5 as a substitute for the catalyst in the second reactor.

### Example 55

In another example, catalytic reaction was also carried out in the quartz-tube reactor (i.d. 1.50 cm, length 60 com) at 660° C., packed with 5.000 g catalyst, but with reactant flows: 20.0 mL/min of methane, 5.0 mL/min of oxygen, 6.0 mL(liquid)/h of 40 wt % HBr/H₂O solution. The products were analyzed by a gas chromatography. Methane conversion rate was 26.7%, and the selectivities of CH₃Br, CH₂Br₂, CO and CO₂ were 82.2%, 3.3%, 11.9% and 2.6%, respectively. The composite undergone first step reaction was directly introduced into glass-tube reactor (i.d. 1.5 cm) at 240° C., which was packed with 8.0 g 14.0 wt % MgO/HZSM-5 catalyst. The final products were analyzed by a gas chromatography. The conversions rate of CH₃Br and CH₂Br₂ were 100% through the second reactor and the products were hydrocarbons of C2˜C13.

### Example 56

CO is the main by-product in first step reaction and it is difficult to separate from CH₄. So CO and CH₄ were returned into first reactor for further reaction without separation. CH₄, O₂, CO (N₂ as internal standard) and 40 wt % HBr/H₂O (6.0 mL/h) were fed together into the first reactor, with flows: 15.0 mL/min of CH₄, 5.0 mL/min of O₂, 3.0 mL/min of CO, 5.0 mL/min of N₂, 6.0 mL/h of 40 wt % HBr/H₂O (liquid). The reaction was carried out at 660° C. and the conversion rate of methane was 30.4%, the selectivities of CH₃Br, CH₃Br₂ and CO₂ were 86.5%, 1.7% and 11.8%, respectively. The total selectivity of CH₃Br and CH₃Br₂ was 88.2%. The composite through first reaction was directly introduced into the second reactor in which CH₃Br and CH₃Br₂ were all converted into hydrocarbons of C₂˜C₁₃.

We claim:

1. A process comprising:
(a) reacting methane, oxygen and HBr/H₂O over a first catalyst in a first reactor to form CH₃Br and CH₂Br₂;
(b) converting CH₃Br and CH₂Br₂ into C₃˜C₁₃ hydrocarbons and HBr over a second catalyst in a second reactor; and
(c) recovering the HBr produced in step (b)

2. The process of claim 1, wherein the first catalyst consists of metals or non-metals or compounds thereof.

3. The process of claim 1, wherein the second catalyst is metal oxide supported on HZSM-5 or metal halide supported on HZSM-5.

4. The process of claim 2, wherein the first catalyst comprises one or more compounds of metals or non-metals selected from the group consisting of Ru, Rh, Pd, Ir, Pt, Fe, Co, Ni, Cu, Zn, Mg, Ca, Sr, Ba, Sc, Y, La, Ce, Ti, Zr, V, Nb, Ta, Cr, Mo, W, Mn, Re, Ag, Au, Cd, Al, Ga, In, Tl, Si, B, Ge, Sn, Pb, Sb, Bi, Te, Pr, Nd, Sm, Eu, Gd, and Tb.

5. The process of claim 2, wherein step (a) is carried out in a fixed-bed reactor at a temperature between about 400° C. and about 800° C., and pressure between about 0.5 atm and about 10.0 atm.

6. The process of claim 3, wherein the second catalyst comprises one or more HZSM-5 supported oxide or halide of metals or non-metals selected from the group consisting of Ru, Fe, Co, Ni, Cu, Zn, Mg, Ca, Sr, Ba, Sc, Y, La, Ce, Ti, Zr, V, Nb, Ta, Cr, Mo, W, Mn, Cd, Al, Ga, In, Tl, Si, Ge, Sn, Pb, Sb, Bi, Pr, Nd, Sm, Eu, Gd and Tb.

7. The process according to the claim 3, wherein step (b) occurs at a temperature between about 150° C. and about 500° C., and a pressure between about 0.5 atm and about 50 atm.

8. The process of claim 1, wherein HBr recovered in step (c) is recycled into the first reactor.
